(19) United States
(12) Patent Application Publication
(10) Pub. No.: US 2022/0304582 A1
(43) Pub. Date: Sep. 29, 2022

(54) TERRITORY MAPPING IN PSEUDO-CONTINUOUS ARTERIAL SPIN LABELING

(71) Applicants: Siemens Healthcare GmbH, Erlangen (DE); The General Hospital Corporation, Boston, MA (US); The Trustees of The University of Pennsylvania, Philadelphia, PA (US)

(72) Inventors: Yulin Chang, Belmont, MA (US); Jason Stockmann, Cambridge, MA (US); Thomas Witzel, Boston, MA (US); Marta Vidorreta Diaz De Cerio, Pamplona (ES); John Detre, Philadelphia, PA (US); Lincoln Craven-Brightman, Charlestown, MA (US)

(21) Appl. No.: 17/656,660

(22) Filed: Mar. 28, 2022

Related U.S. Application Data
(60) Provisional application No. 63/167,182, filed on Mar. 29, 2021.

(51) Int. Cl. A61B 5/026; A61B 5/00; G01R 33/54; G01R 33/3415

(52) U.S. Cl. CPC A61B 5/0263; A61B 5/0042; G01R 33/54; G01R 33/3415

(57) ABSTRACT

Systems and methods for localized pseudo-continuous ASL (pCASL) of arterial blood local multi-coil arrays in an MRI system allow a series of pulses to selectively label blood with an on-resonance magnetic field in one or more arteries in a labeling plane while masking blood in others with an off-resonance magnetic field. This allows perfusion imaging and is well suited for imaging of cerebral blood flow.

### CROSS REFERENCE TO RELATED APPLICATIONS

This application claims the benefit of U.S. Provisional Application 63/167,182 filed Mar. 29, 2021, which is incorporated herein by reference in its entirety.

### TECHNOLOGY FIELD

The present invention relates generally to methods, systems, and apparatuses for spin labeling fluids in MRI imaging applications. The techniques described herein may be used, for example, to selectively label or enhance labeling blood in a subset of arteries by creating an inhomogeneous field in a labeling plane.

### BACKGROUND

Arterial spin labeling (ASL) MRI is a noninvasive technique for measuring cerebral blood flow (CBF). Perfusion-weighted color maps and relative cerebral blood flow (relCBF) color maps can be calculated. Exemplary uses of ASL include 2D imaging based on an EPI sequence enhanced for ASL and 3D imagining using a turbo gradient spin echo technique and an ASL preparation. Labeling of arterial blood can be achieved with a number of different strategies. Continuous ASL (CASL) attempts to continuously invert or saturate blood as it passes a particular plane. Pulsed ASL (PASL) employs a single pulse to define a volume containing arterial blood for labeling.

Pseudo-continuous ASL (pCASL) is the most widely used ASL implementation because it affords high labeling efficiency (SNR). pCASL uses a narrow labeling plane through which flow-related adiabatic inversion of arterial spins occurs. The tagging is usually performed immediately proximal to the imaging volume, which minimizes signal loss from the decay of labeled blood. In pCASL, magnetization of arterial blood water is inverted (i.e., ‘labeled’) using a long train of short, Hanning-windowed block pulses. The labeling efficiency of pCASL depends on several subject-specific factors, including off-resonance of the B₀ field in the labeling plane.

The challenge of off-resonance B₀ fields is exacerbated in pCASL because the scanner shim usually prioritizes the field homogeneity of the imaging volume in the brain over that of the labeling plane. Additional explanation of exemplary prior art ASL/pCASL techniques can be found in Dai et al., “Continuous Flow-Driven Inversion for Arterial Spin Labeling Using Pulsed Radio Frequency and Gradient Fields,” Mag. Res. In Med. 60:1488-1497 (2008) and Alsop et al., “Recommended Implementation of Arterial Spin-Labeled Perfusion MRI for Clinical Applications: A Consensus of the ISMRM Perfusion Study Group and the European Consortium for ASL in Dementia,” Mag. Res. In Med. 73:102-116 (2015).

Some prior methods have attempted to provide a calibration scan for off-resonance in the labeling plane or to adjust the labeling RF pulse to improve labeling efficiency. See, e.g., Jung et al. MRM 64(2010):799, Jahanian et al. NMR Biomed 24(2011):1202, Chen et al. MRM 77(2017):1841, Luh et al. MRM 69(2013):402. A recent method was proposed that attempts to optimized Bi and gradient in unbalanced pCASL (ubpCASL) to improve the labeling efficiency for off-resonance blood (Zhao et al. MRM 78(2017):1342).

None of these methods directly addresses the underlying problem of B₀ field off-resonance at the labeling plane. In addition, they only improve labeling efficiency for the whole labeling plane and do not provide control over labeling efficiency for blood in individual arteries. There remains a need for more efficient and targeted inversion and, thus, higher sensitivity and resilience to artifactual hypoperfusion that is otherwise due to lower labeling efficiency.

### SUMMARY

The present disclosure generally relates to MRI spin labeling techniques where a magnetic shim array is used to shim the magnetic field in the labeling plane by application of a set of DC currents. The resulting magnetic field can shape the labeling efficiency of an RF labeling field to improve labeling efficiency and/or reduce labeling efficiency in certain regions of the patient anatomy to selective and improve perfusion imaging of blood passing through one or more selected arteries.

In one embodiment, the present disclosure is directed to A method for perfusion magnetic resonant imaging (MRI) within an MRI system having at least one RF coil configured to transmit an RF signal. Steps includes providing a coil array configured for placement on a patient in the MRI system at a labeling plane that bisects a plurality of arteries, capturing one or more first MRI images of patient anatomy at a first imaging plane that is different from the labeling plane. Steps further include applying a series of RF pulses to the at least one RF coil, to create an RF labeling field in the labeling plane and applying a first set of direct currents to a plurality of coils in the coil array while the RF pulses are applied. A first resulting field in the labeling plane is a non-uniform magnetic field such that the RF field labels the nuclear spin of blood in at least one artery of the plurality of arteries, while substantially not labeling the nuclear spin of blood in a remainder of the plurality of arteries. The method further includes capturing one or more second MRI images of patient anatomy at the first imaging plane after a perfusion delay and comparing the one or more first and second MRI images to generate a map of perfusion associated with the at least one artery. This method can be repeated any number of suitable times.

In one aspect, the perfusion delay is 1-3 seconds after the step of applying a series of RF pulses. In another aspect, the steps of capturing one or more first MRI images, applying a series of RF pulses, applying a set of direct currents and capturing one or more second images are repeated for a second imaging plane. In another aspect, the coil array is a device placed on the patient's head or a device placed on the patient's neck.

In one aspect, the non-uniform magnetic field includes one or more regions where nuclear spins are at least 100 Hz off-resonance of the RF labeling field at the location of the remainder of the plurality of arteries. In another aspect, steps include applying a second set of direct currents to the plurality of coils in the coil array, such that a second resulting field in the first imaging plane is a magnetic field that is more uniform than without the second set of DC currents. In another aspect, the labeled artery includes a carotid artery. In another aspect, the labeled artery includes a vertebral artery.

In another embodiment, an MRI system includes at least one RF coil configured to transmit an RF signal, a coil array configured for placement on a patient in an MRI imaging system at a labeling plane that bisects a plurality of arteries, and a computer. The computer is configured to implement the methods described above.

In another embodiment, a method for perfusion magnetic resonant imaging (MRI) within an MRI system having at least one RF coil configured to transmit an RF signal includes providing a coil array configured for placement on a patient in the MRI system at a labeling plane that bisects a plurality of arteries and capturing one or more first MRI images of patient anatomy at a first imaging plane that is different from the labeling plane. The method further includes applying a series of RF pulses to the at least one RF coil, to create an RF labeling field in the labeling plane, and applying a first set of direct currents to a plurality of coils in the coil array while the RF pulses are applied, such that a first resulting field in the labeling plane results in a labeling region where a nuclear spin of blood is more on-resonance with the RF labeling field than without the first set of DC currents. The method further includes capturing one or more second MRI images of patient anatomy at the first imaging plane after a perfusion delay and comparing the one or more first and second MRI images to generate a map of perfusion.

According to one aspect, the first resulting field in the labeling plane includes one or more regions where nuclear spins are at least 100 Hz off-resonance of the RF labeling field.

### FIGURES

The accompanying drawings, which are incorporated in and form a part of the specification, illustrate the embodiments of the invention and together with the written description serve to explain the principles, characteristics, and features of the invention. In the drawings:

FIG. 1A is a perspective view of coil arrangements and an image of a front view of an exemplary cranial/brain multi-coil shim coil array device for use with some embodiments;

FIG. 1B is two side views of an exemplary cervical/neck multi-coil shim coil array device for use with some embodiments;

FIG. 2 is a diagram of the relative relationship of the field regions of exemplary multi-coil shim coil array devices and an exemplary labeling plane for use with some embodiments;

FIG. 3 is a timing diagram of an exemplary labeling and imaging process for use with some embodiments;

FIG. 4A is an example of the labeling field and imaging results obtained when operating an exemplary cranial/brain multi-coil shim coil array device in accordance with one embodiment of territory mapping with pCASL;

FIG. 4B is an example of the labeling field results obtained when operating an exemplary cervical/neck multi-coil shim coil array device in accordance with one embodiment of territory mapping with pCASL;

FIG. 5 is a flowchart of exemplary operation of an embodiment of territory mapping with pCASL; and

FIGS. 6A and 6B are system diagrams of exemplary computing and MRI imaging systems for used with some embodiments of territory mapping with pCASL.

### DESCRIPTION

This disclosure is not limited to the particular systems, devices and methods described, as these may vary. The terminology used in the description is for the purpose of describing the particular versions or embodiments only, and is not intended to limit the scope.

As used herein, the terms “algorithm,” “system,” “module,” “engine,” or “architecture,” if used herein, are not intended to be limiting of any particular implementation for accomplishing and/or performing the actions, steps, processes, etc., attributable to and/or performed thereby. An algorithm, system, module, engine, and/or architecture may be, but is not limited to, software, hardware and/or firmware or any combination thereof that performs the specified functions including, but not limited to, any use of a general and/or specialized processor in combination with appropriate software loaded or stored in a machine-readable memory and executed by the processor. Further, any name associated with a particular algorithm, system, module, and/or engine is, unless otherwise specified, for purposes of convenience of reference and not intended to be limiting to a specific implementation. Additionally, any functionality attributed to an algorithm, system, module, engine, and/or architecture may be equally performed by multiple algorithms, systems, modules, engines, and/or architectures incorporated into and/or combined with the functionality of another algorithm, system, module, engine, and/or architecture of the same or different type, or distributed across one or more algorithms, systems, modules, engines, and/or architectures of various configurations.

Methods and apparatus described herein use local shim coils to adjust the B₀ field for an individual artery (or arteries) or the whole plane at the labeling location, so that the labeling efficiency related to the off-resonance effect on blood passing through the artery can be precisely controlled. Using shim coils in close proximity to arteries can also selectively affect field homogeneity local to the labeling plane and can allow for more efficient inversion and, thus, higher sensitivity and resilience to artifactual hypoperfusion due to selectively affecting labeling efficiency.

Local control of the B₀ field at the labeling plane can allow for mapping of perfusion flow territories without modification to the pulse sequence used in the art. By controlling individual coils with DC currents to create regions of anatomy that are more on resonance or more off-resonance with the B₀ field, labeling efficiency can be tailored to a desired territory map (e.g. the labeling efficiency can be localized to be high or low by applying predetermined DC signals to individual coils to create a predetermined heterogeneous field). One exemplary application includes selectively labeling blood in different arteries (by enhancing the labeling efficiency), while masking others by reducing the labeling efficiency to near zero. Embodiments provide a method of controlling the B₀ field at the labeling plane using a local multi-coil (MC) B₀ shim array. To demonstrate its effect on modulating pCASL labeling efficiency, flow territory maps of CBF can be obtained by specifically controlling the B₀ fields near specific arteries by selectively controlling individual coils of a subset of coils in a B₀ shim array. One exemplary shim array can be found in U.S. Pat. No. 10,261,145, which is designed to be worn on the patient's head. Other embodiments, such as a cervical B₀ shim array for application to a patient's neck for arterial labeling therein.

The concepts of on resonance and off resonance are understood in the art. When a B₀ field is said to be on resonance, it means that the Larmor frequency (resulting from that magnetic field) of the nuclear spin of protons of atoms in the patient anatomy is approximately the same as the frequency of the RF labeling field. This field is generated by applying RF signals to an RF transmit coil (e.g., main body coil) in the MRI. This means that those protons can be labeled by the RF field such that the nuclear spin of those atoms is changed (e.g., flipped) by the RF labeling field. When a B₀ field is off resonance, the Larmor frequency of the nuclear spin of protons of atoms in the patient anatomy is different from the frequency of the RF labeling field. This means that those off-resonant nuclear spins will not be substantially changed by the resonance of the RF field, meaning that those atoms will not have a spin that is labeled by the RF labeling field. Thus, a DC magnetic field causes atoms in the anatomy to be spin labeled or not. We can refer to this bifurcation as changing the labeling efficiency of the RF field. In regions where the labeling efficiency is high, the B field is on resonance such that most atoms have their nuclear spin changed/flipped by the RF field; in regions where the labeling efficiency is low, most atoms do not have their spin changed by the RF field. As used herein, a region of anatomy is said to be substantially on-resonance when the Larmor frequency of the nuclear spin is within 25 Hz of the RF field that is applied to label the spin. As used herein a region of anatomy is said to be substantially off-resonance when the Larmor frequency of the nuclear spin is more than 100 Hz different from that of the RF field that is applied to label the spin. Blood in regions that are in a region of anatomy that is substantially off resonance is said to not be substantially labeled by the RF field because the labeling efficiency is low, at least less than 10%. Blood in regions that are in a region of anatomy that is substantially on resonance is said to be substantially labeled by the RF field because the labeling efficiency is high, at least less than 80%. In some embodiments labeling efficiency above 90% can be achieved using the techniques described herein.

Some embodiments use a dedicated cervical ASL MC shim array (for example, with 7-turn loops) designed for improved field control efficiency and flexibility in the ASL labeling region. This can be placed in the labeling plane by placing on the patient's neck.

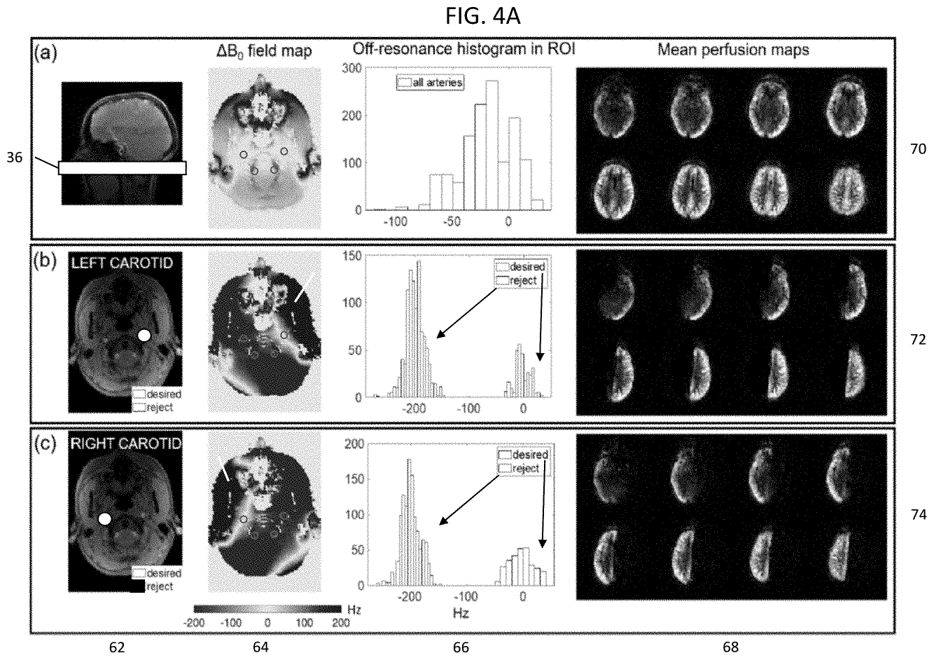

MC B₀ shim arrays can use an array of independently driven loops (typically arranged on three-dimensional surface) of that are placed in close proximity to patient tissue to generate nonlinear, rapidly switchable B₀ offsets in the body. MC B₀ shim arrays have been used in the art for improving B₀ homogeneity to reduce artifacts in imaging and spectroscopy. Embodiments extend the utility of MC shim arrays to improve ASL robustness and enable flow territory mapping by instead intentionally creating a predetermined field heterogeneity. In one exemplary proof-of-concept experiment, a 32-channel “AC/DC” array with single-turn loops is used both for RF receive and for carrying DC currents (shown in FIG. 3). Exemplary pCASL experiments were performed on a MAGNETOM Prisma 3T MRI scanner (Siemens Healthcare, Erlangen) using the scanner body coil as the transmit coil. FIG. 3, explained below, shows a block diagram of the labeling, saturation, and spatial encoding blocks of the pCASL sequence, indicating the points in time when the MC shim field is switched on and off.

Prior art pCASL flow territory mapping techniques generally involve either the use of RF phase modulation to acquire images with different labeling efficiency distributions or rotating in-plane gradients during labeling. In contrast, the approach used by exemplary embodiments does not require modifications to the standard pCASL imaging sequence or advanced postprocessing strategies. Further, the ability to shim B₀ homogeneity at the labeling plane can improve the labeling efficiency of nonselective pCASL as well as the labeled arteries in selective pCASL. This approach can have a particular impact for 7T pCASL, where off-resonance may pose a severe obstacle to reproducible ASL.

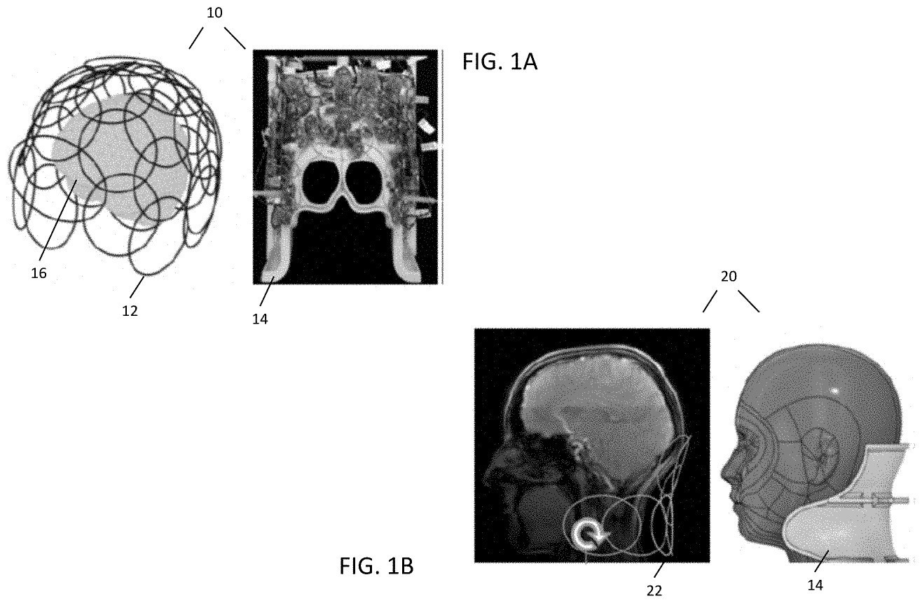

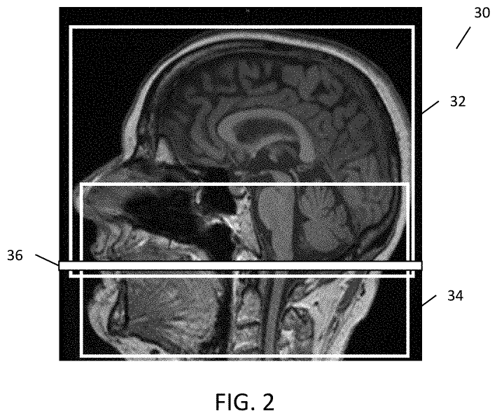

FIGS. 1A-1B shows the schematics and photo/rendering of such local shim coils for brain (1A) and neck (1B). Cranial/brain shim coil device 10 is a wearable coil device that includes and an array of shim coils 12 and housing body 14 that can be wrapped, molded, placed, worn, or otherwise temporarily affixed to the patient's head (generally referred to herein as wearable while the patient is in an MRI imaging system), such that coils 12 partially surround brain 16. In one embodiment, the coils can be integrated into a flexible wrap-around body; in another embodiment, coils are rigidly coupled to a helmet body (such as housing body 14, which may be molded or 3D-printed) worn by the patient. Preferably coils 12 include at least a subset of coils that extend below the brain to allow labeling of arterial blood before it reaches the brain (e.g. in the cervical region). In one embodiment, shim coils 12 include an array of 32 seven-turn coils that are arranged three-dimensionally around the patient's brain in close proximity to (or touching) the patient's head when cranial/brain shim coil device 10 is worn. It should be appreciated that the coil arrays discussed herein need not have rows or columns per se and that any suitable geometric arrangement of coils arranged on a three-dimensional surface may be used to create the array. Using a MC shim array (shim coils 12) can successfully territory map fields when labeling blood to image perfusion by the right and left internal carotid arteries, but can less successfully target the vertebral arteries in some embodiments.

FIG. 1B shows a cervical/neck shim coil device 20, which is also a wearable device that includes an array of shim coils 22 and a body 24. Body 24 can be constructed and be similar to a cervical collar stabilization device. This allows the cervical/neck shim coil device 20 to be rigidly affixed to a patient's neck area. In some embodiments, the cervical/neck shim coil device 20 is worn on the back of the patient head, making it more comfortable than a stabilization device. In one embodiment, shim coils 22 include an array of twelve seven-turn coils that are arranged three-dimensionally around the patient's cervical region in close proximity to (or touching) the patient's neck when cervical shim coil device 20 is worn. By providing a MC shim array directly in the cervical region, as shown in FIG. 1B, stronger fields can be obtained at the labeling plane that intersects the carotid and vertebral arteries, allowing successfully target any of the carotid or vertebral arteries. Similarly, a cervical shim coil device allows for a greater range inferior labeling planes, which may be advantageous.

Wearable coil devices, such as cranial/brain shim coil device 10 or neck/cervical shim coil device 20, can combine RF and DC shim coils. Brain shim coil device 10 has been used as a magnetic shim device in other applications to improve the B₀ field homogeneity or to intentionally introduce heterogeneity in certain regions to mask spin labeling. By applying DC current to individual coils 12 and 22, the B₀ field created by the MRI magnet can be manipulated as desired (more homogenous or less homogenous or to improve/decrease resonance in certain regions to manipulate labelling efficiency). Thus, by using an array of coils, in addition to improving the B₀ field homogeneity and conventional labeling efficiency, the high degrees of freedom (from the high count of shim coils in the array) allow embodiments to achieve localized territory mapping of the blood perfusion where the labeling of blood in some arteries is nearly turn off while others are unaffected.

These coils can also be used as RF sensors to assist in collecting imaging information to detect resonance in response to stimuli provided by the RF coil(s) of the MRI machine during an imaging phase. Accordingly, it should be understood that coils 12 and 22 can provide magnetic shimming, target spin labeling, and RF sensing functions during MRI operation.

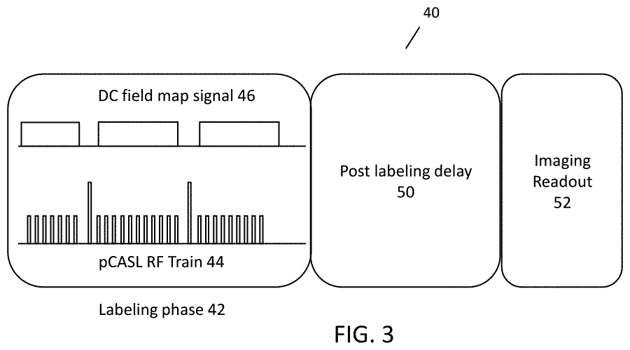

In FIG. 2, patient image of a lateral head view 30 of a patient MRI with illustrative information of how wearable shim coil devices can affect imaging, including perfusion imaging. Region 32 is the primary field region that can be controlled by coils 12 in a brain shim coil device 10. While most of this region encompasses the brain, the coils can also control local fields in the top of the cervical region, allowing ASL in that region. Region 34 is the primary field region that can be controlled by coils 22 in a brain shim coil device 20. This region encompasses more of the cervical region, allow ASL at more possible locations for perfusion imaging. Labeling plane 36 is an exemplary labeling plane that can be used for ASL to accomplish perfusion imaging in the patient brain. A labeling plane used in ASL bisects at least one artery such that blood flowing through the plane can be labeled.

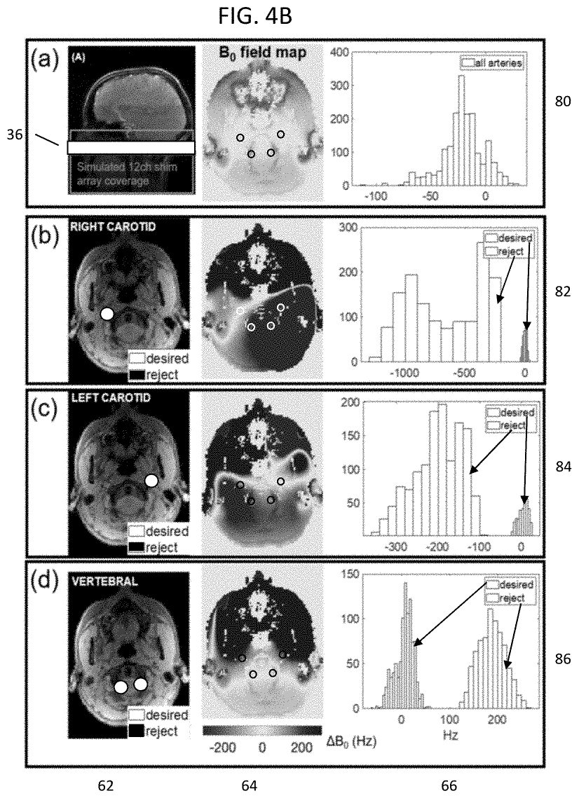

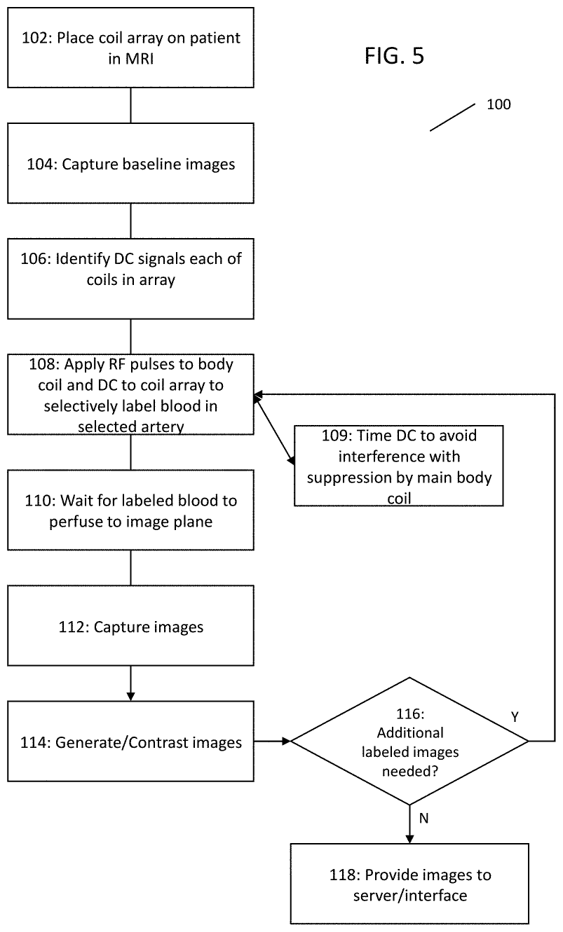

An exemplary acquisition used an embodiment of a pCASL sequence with 1.8s labeling duration (between 1 and 2 seconds), 1.8s post-labeling delay (between 1 and 3 seconds, nominal), 4-pulse background suppression, single-shot 3D stack-of-spirals readout with 2 spiral interleaves for each partition, spatial resolution of 3.75 mm isotropic, TR/TE=5000/10.3 ms, eight control-label pairs, and scan time of 1 min 44 sec Time of flight (TOF) images were acquired and segmented during the scan to obtain vessel masks for the two carotid and two vertebral arteries. For territory mapping (such as shown in FIGS. 4A-4B), one carotid is marked as “desired” while the other three vessels are masked as “reject.”

It is known that pCASL labeling efficiency falls off rapidly for B₀ shifts over ˜100 Hz off-resonance. To selectively shift spins in localized regions outside this pCASL “passband,” some embodiments use a convex objective function with a fast solver, known in the art (such as a MOSEK solver engine). This exemplary technique allow for available degrees of freedom to shift the “reject” voxels while holding the “desired” voxels on-resonance according to a localized territory map.

One exemplary experiment used to demonstrate an exemplary embodiment, uses a pipeline having 1 min 20 sec. for TOF image acquisition, 2 minutes for data transfer and masking, and 10 seconds for the field solver. Exemplary experiments and calculations demonstrate that by shimming the field at the left carotid to such that the labeling in the area is about 200 Hz off resonance, the labeling in that artery is essentially nulled, leading to nearly no perfusion measured in the right brain in exemplary scans.

Another advantage of using local shim coils is the ability to dynamically turn on and off shim during scan, which ensures that the technique does not interfere with imaging of the brain or the background suppression for reduced noise. FIG. 3 illustrates exemplary control signals that can be applied to the conventional main RF coil(s) in an MRI system and the coils in the local shim coil arrays to selectively control labeling efficiency to label or mask blood flowing through different arteries to provide perfusion map imaging. The pulses that create these fields are applied by the main RF coil(s) and can include can include background suppression pulses. Meanwhile the territory mapping of the labeling field efficiency is accomplished by applying a set of DC signals to the coils in the MC shim coils array. In some embodiments, the DC shim signal is paused while the main body RF coil in the MRI machine is activated to supply background suppression pulses, which would interfere with the shim field. The DC signals to the MC shim array coils can also provide shimming in or out of the labeling plane to improve imaging quality during an imaging phase.

FIG. 3 illustrates the phases 40 used in pCASL imaging with regional territorial mapping used in some embodiments. In a spin labeling phase 42, arterial blood is selectively labeled by applying a pCASL RF train 44 to the main RF coil(s) of the MRI system. This provides a pCASL labeling that will label molecules in the labeling plane in accordance with pCASL techniques of the prior art. The labeling efficiency of this pulse train is determined by a heterogeneous field created by an MC shim array. A processor calculates the DC signals needed to create a B₀ field map in the labeling plane using a solver engine (such as a quadratic solver). DC currents 46 are determined for each coil in the MC coil array can then be applied to each coil during the pCASL labeling phase. This results in a net field in any given location the labeling plane that can be more or less on resonance than the expected field from pCASL RF train 44 from the main RF coil(s). This allows DC currents 46 to shape the labeling efficiency in the labeling plane, created a net on-resonance field to anatomy targeted for labeling and a net off-resonance field (e.g., using>+/−100 Hz out of phase fields, +/−200 Hz nominal) to anatomy targeted for masking. This is well suited to labeling and suppressing labeling of blood in arteries in the cervical region, but can be used for a wide variety of vascular anatomy for various perfusion imaging applications.

In some embodiments, pCASL RF train 44 uses a series of Hanning-windowed block pulses having magnitude and rate consistent with prior art labeling methods, tailored to the anatomy and to the RF coil(s) in the MRI system being used. DC currents 46 represent a set of DC signals applied to the coils in the array where different coils in the shim array receive different DC currents 46, each having a predetermined magnitude (but substantially the same timing). In addition to the DC currents 46 during the labeling phase, in some embodiments, similar DC currents can also be supplied be individual coils in the MC shim array during the imaging readout phase 52 to provide background field shimming to improve imaging homogeneity in the imaging plane.

During conventional operation of an MRI, the main RF body coil for the machine (which is separate and distinct from the shim coil arrays describe herein) will provide a few background field suppression pulses (the spikes in pCASL RF train 44). These pulses can be detrimentally affected by operating the shim coils with a pCASL during these pulse windows. Accordingly, DC currents 46 can have gaps coinciding to background field suppression pulses in RF train 44 to avoid interference. The DC shim fields are thus paused during those pulses.

Once pCASL RF train 44 has labeled the chosen arterial blood (which takes around 1.8s in some embodiments), a post labeling delay 50 allows the labeled blood to perfuse from the labeling plane to the brain tissue, which can then be imaged (e.g. a 1-2 second delay in some embodiments) during imaging readout phase 52. Any suitable imaging technique can be used, such as a stack of spirals technique. The fields are applied during labeling and then zeroed out during saturation and readout sequence modules.

FIGS. 4A and 4B illustrate some observed and calculated results using the technique shown in FIG. 3. FIG. 4A shows the results when labeling with cranial/brain shim coil device 10; FIG. 4B shows the results when labeling with cervical/neck shim coil device 20. Column 62 is a mapping of the targeted anatomy in the labeling plane. White circles indicate the artery selected for labeling. Column 64 is the theoretical field map created by the pCASL RF train within the anatomy when applied to a selected subset of coils within a wearable shim coil array. Darker regions indicate that the field is off-resonance and consequently has poor labeling efficiency in the region. Masked arteries should fall in darker regions. Lighter regions indicate that the field is on-resonance and consequently that labeling efficiency is high. Arteries selected for labeling should fall in lighter regions. Column 66 is a histogram of the field resonance applied to selected anatomy. Note that regions fall into on-resonance and off-resonance buckets centered at −0 Hz and >+/−200 Hz (at least >+/−100 Hz), respectively. Column 68 shows the resulting images taken in-vivo on a test subject using the technique.

Turning to FIG. 4A, which shows the results using cranial/brain shim coil device 10 for selective pCASL. Row 70 shows a baseline for pCASL imaging in image plane 36 using cranial/brain shim coil device 10. In this case, no territory mapping is used and all arteries are targeted with high labeling efficiency on-resonance. The resulting perfusion images show that blood reaching both hemispheres is labeled. Row 72 shows an example where the left carotid artery is select for spin labeling and the remaining arteries are masked. As can be seen in the resulting in-vivo images, perfusion is mapped to a single hemisphere. Row 74 shows the same technique applied to the right carotid.

FIG. 4B shows the results using cervical/neck shim coil device 20 for selective pCASL. Row 80 shows a baseline for pCASL imaging in image plane 36 using cervical/neck shim coil device 20. In this case, no territory mapping is used and all arteries are targeted with high labeling efficiency on-resonance. The resulting perfusion images show that blood reaching both hemispheres is labeled. Row 82 shows an example where the left carotid artery is select for spin labeling and the remaining arteries are masked. Perfusion should be mapped to a single hemisphere. Row 84 shows the same technique applied to the right carotid. Because of the proximity to the cervical spine, cervical/neck shim coil device 20 is also well suited for territory mapping of vertebral arteries. Row 86 shows the field mapping used to label the vertebral arteries and mask carotid arteries.

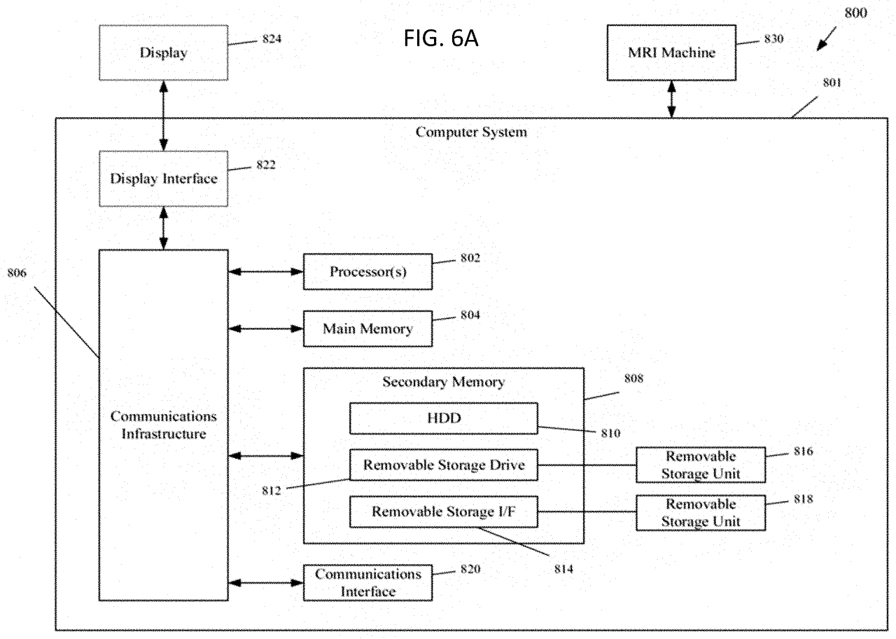

FIG. 5 is a flowchart of an exemplary method 100 for providing territory mapped pCASL using a wearable shim coil array. At step 102, the wearable shim coil array is placed in contact with the patient as in an MRI system. This can include strapping, affixing, resting, securing the shim coil array device to the patient or using any other suitable method, such as placing a rigid shim coil array on the bed of the MRI system such that it cradles the patient head or neck (or other anatomy if used for applications other than brain perfusion). These applications of the coil device can be collectively referred to as placing the device on the patient.

At step 104, the MRI system is used to capture one or more baseline MRI images of patient anatomy at a first location different from the labeling plane without any spin labeling. These can be used to assist in selecting the signals determined in step 106 (such as by helping improve location of arteries, determining inhomogeneities, etc.) or can be used as a baseline image to contrast pCASL images allowing perfusion to be mapped. In some embodiments, DC fields can be applied to the coils in the MC shim coil array to shim the field in the imaging plane during this step.

At step 106, a processor determines the set of DC currents 46 for each coil in the array that is needed to achieve the desired territory mapping for pCASL using a field solver algorithm. The set of DC currents generally includes different values for each coil in the array. This step can also include selecting frequency, duration, and magnitude of the pCASL RF pulses to be applied to the main RF coil(s) of the rest of the MRI system (such as those in pCASL RF Train 44). The summed field created by the different DC signals to the MC shim coils and the RF signals to the main RF coils(s) of the MRI system can result in a targeted part of patient anatomy to be in resonance with the resulting in an improved labeling field, while other parts may be off resonance, causing a masking effect. In some embodiments, the order of these steps 104 and 106 can be reversed (or step 104 can be repeated), particularly where the field solver used takes several seconds to process.

At step 108, the selected DC signals are applied to the individual coils of the shim coil array (while the pCASL RF train is applied to the RF coil(s) of the MRI system for labeling.). This allows conventional labeling techniques to be used for RF spin labeling by the MRI machine while the MC shim coil array shapes the labeling efficiency. In some embodiments, step 108 applies determined DC signals to the coils in the wearable array during the pCASL labeling process to create a non-uniform resonant magnetic field that spin labels blood in at least one artery of the plurality of arteries, while masking labeling of blood in a remainder of the plurality of arteries. This step can include a related step 109 where the DC currents in the shim coil array are paused while a main body coil of the MRI applies background field suppression pulses. At the completion of step 108, arterial blood in a selected subset (one or more) of arteries in the labeling plane is spin labeled, while blood in the remaining arteries in the labeling plane is not spin label (due to the off-resonance field in the relevant regions). In some embodiments, this process takes 1-2s, 1.8s nominal.

At step 110, the method 100 waits for labeled blood to perfuse from the labeling plane to a desired image plane (such as one intersecting the hemispheres of the patient brain). In some embodiments, this wait time is also 1-2 s, 1.8 s nominal, but can be selected based on the expected perfusion time.

At step 112, the MRI system captures post-perfusion MRI images of patient anatomy using any conventional technique, such as spiral imaging. This can be the same imaging technique used in step 104 and should be done at the same location. In some embodiments, spiral imaging can be used. In some embodiments, one or more coils in the wearable coil array can be used during imaging at step 112 or 104 to improve field homogeneity in the imaging plane or as RF receivers to receive imaging signals. To improve field homogeneity, DC signals for the coils in the MC shim array can be calculated and applied in a manner substantially similar to those in steps 106 and 108. The important distinction from those signaling steps is that the set of DC signals optionally applied as part of step 112 is calculated and applied to the MC shim array for the purpose of shimming field homogeneity across the imaging plane rather than creating a targeted field inhomogeneity across the labeling plane.

At step 114, the image data captured in step 112 is contrasted (or otherwise compared algorithmically) to the image data captured at step 104. This can emphasize the presence and absence of spin-labeled perfusion in the resulting image. This step generates a map of perfusion associated with the labeled artery/arteries. This map can be generated by any suitable algorithm in the art.

At step 116, a processor or operator determines if additional spin-labeled images are needed. For example, additional image slices may be desired or additional territory maps of other arteries may be needed. If so, method 100 repeats, starting at step 104 (or optionally at step 106 if additional baseline images are not needed).

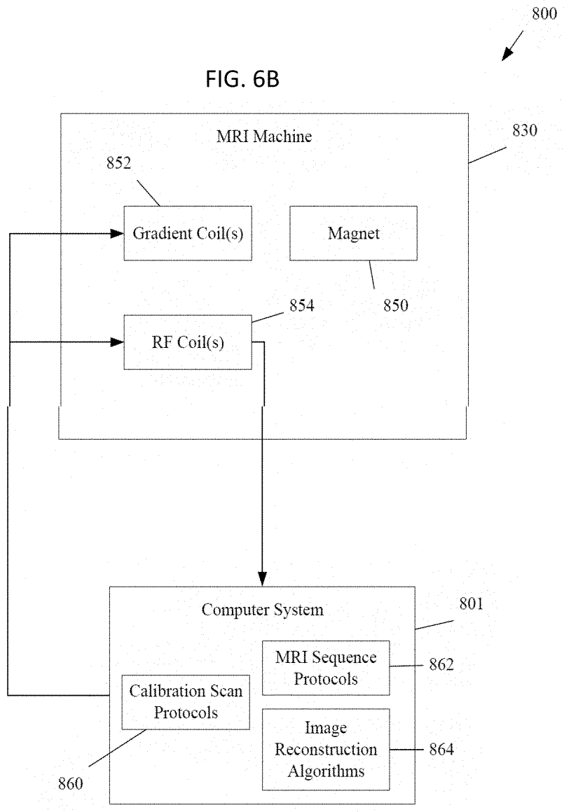

The method of FIG. 5 can be carried out by the system shown in FIGS. 6A-B, such as at the direction of computer system 801. Each signal used in method 100 (or those shown in FIG. 4) can be generated by any suitable drive circuit operating under processor control.

Once all images have been collected, step 118, the image data can be provided to whatever image server, data store, cloud application, or user interface is suitable for analysis and presentation of the spin-labeled images.

In some embodiments, the systems and techniques described above can be implemented in or by a medical imaging system, such as the medical imaging system 800 illustrated in FIGS. 6A and 6B.

FIG. 6A is an architecture diagram of medical imaging system 800 that may be used in some embodiments. As noted above, the medical imaging system 800 can include a computer system 801 and an imaging machine 830 (e.g., an MRI machine). The computer system 801 may include one or more processors 802. Each processor 802 is connected to a communication infrastructure 806 (e.g., a communications bus, cross-over bar, or network). The processor(s) 802 can include a CPU, a GPU, an AI accelerator, and/or a variety of other processor types. Computer system 801 may include a display interface 822 that forwards graphics, text, and other data from the communication infrastructure 806 (or from a frame buffer, not shown) for display on the display unit 824.

Computer system 801 may also include a main memory 804, such as a random-access memory (RAM), and a secondary memory 808. The secondary memory 808 may include, for example, a hard disk drive (HDD) 810 and/or removable storage drive 812, which may represent a floppy disk drive, a magnetic tape drive, an optical disk drive, a memory stick, or the like as is known in the art. The removable storage drive 812 reads from and/or writes to a removable storage unit 816. Removable storage unit 816 may be a floppy disk, magnetic tape, optical disk, or the like. As will be understood, the removable storage unit 816 may include a computer readable storage medium having tangibly stored therein (embodied thereon) data and/or computer software instructions, e.g., for causing the processor(s) to perform various operations.

In alternative embodiments, secondary memory 808 may include other similar devices for allowing computer programs or other instructions to be loaded into computer system 801. Secondary memory 808 may include a removable storage unit 818 and a corresponding removable storage interface 814, which may be similar to removable storage drive 812, with its own removable storage unit 816. Examples of such removable storage units include, but are not limited to, USB or flash drives, which allow software and data to be transferred from the removable storage unit 816, 818 to computer system 801.

Computer system 801 may also include a communications interface 820. Communications interface 820 allows software and data to be transferred between computer system 801 and external devices. Examples of communications interface 820 may include a modem, Ethernet card, wireless network card, a Personal Computer Memory Card International Association (PCMCIA) slot and card, or the like. Software and data transferred via communications interface 820 may be in the form of signals, which may be electronic, electromagnetic, optical, or the like that are capable of being received by communications interface 820. These signals may be provided to communications interface 820 via a communications path (e.g., channel), which may be implemented using wire, cable, fiber optics, a telephone line, a cellular link, a radio frequency (RF) link and other communication channels.

In this document, the terms “computer program medium” and “non-transitory computer-readable storage medium” refer to media such as, but not limited to, media at removable storage drive 812, a hard disk installed in hard disk drive 810, or removable storage unit 816. These computer program products provide software to computer system 801. Computer programs (also referred to as computer control logic) may be stored in main memory 804 and/or secondary memory 808. Computer programs may also be received via communications interface 820. Such computer programs, when executed by a processor, enable the computer system 801 to perform the features of the methods discussed herein. For example, main memory 804, secondary memory 808, or removable storage units 816 or 818 may be encoded with computer program code (instructions) for performing operations corresponding to various processes disclosed herein.

Referring now to FIG. 6B, an exemplary MRI machine 830 can include a magnet 850 (e.g., extending along a bore) that is configured to receive a patient therein and that is configured to produce a generally uniform magnetic field, one or more gradient coils 852 that are configured to produce magnetic field gradients (e.g., linear gradients), and one or more RF coils 854 that are configured to transmit to RF signals to the patient's body and/or receive RF signals therefrom. The computer system 801 (embodiments of which are described in greater detail above) can store and implement calibration scan protocols 860, MRI sequences protocols 862, and/or image reconstruction algorithms 864, as well as a variety of other software modules known in the technical field. The MRI sequence protocols 862 can be embodied as instructions that, when executed by the computer system 801, cause the computer system 801 to control the gradient coils 852 and/or RF coils 854 to apply a particular sequence of magnetic field gradients and/or RF pulses to the patient. The image reconstruction algorithms 864 can be embodied as instructions that, when executed by the computer system 801, cause the computer system 801 to reconstruct an image of the patient based on the RF signal received from the patient (e.g., by the RF coils 854) as caused by the MRI sequence applied thereto. The calibration scan protocols 860 can likewise be embodied as instructions that, when executed by the computer system 801, cause the computer system 801 to apply particular MRI sequences and/or imaging trajectories (e.g., Cartesian or non-Cartesian trajectories) to calibrate the MRI machine 830 for imaging acquisition and/or parameter mapping applications.

The wearable multicoil shim array devices (such as devices 10 and 20) discussed above are included in the RF coils 854 in this system. They can be used as RF imaging coils or operated in a DC shimming mode to improve B₀ homogeneity or to map B₀ inhomogeneity to selectively mask or enhance labeling efficiency of the field created by other RF coils in the RF coils 854.

It is understood by those familiar with the art that the system described herein may be implemented in hardware, firmware, or software encoded (e.g., as instructions executable by a processor) on a non-transitory computer-readable storage medium.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

Aspects of the present technical solutions are described herein with reference to flowchart illustrations and/or block diagrams of methods, apparatuses (systems), and computer program products according to embodiments of the technical solutions. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present technical solutions. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

A second action can be said to be “in response to” a first action independent of whether the second action results directly or indirectly from the first action. The second action can occur at a substantially later time than the first action and still be in response to the first action. Similarly, the second action can be said to be in response to the first action even if intervening actions take place between the first action and the second action, and even if one or more of the intervening actions directly cause the second action to be performed. For example, a second action can be in response to a first action if the first action sets a flag and a third action later initiates the second action whenever the flag is set.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method for perfusion magnetic resonant imaging (MRI) within an MRI system having at least one RF coil configured to transmit an RF signal, comprising:
providing a coil array configured for placement on a patient in the MRI system at a labeling plane that bisects a plurality of arteries;
capturing one or more first MRI images of patient anatomy at a first imaging plane that is different from the labeling plane;
applying a series of RF pulses to the at least one RF coil, to create an RF labeling field in the labeling plane;
applying a first set of direct currents to a plurality of coils in the coil array while the RF pulses are applied, such that a first resulting field in the labeling plane is a non-uniform magnetic field such that the RF field labels the nuclear spin of blood in at least one artery of the plurality of arteries, while substantially not labeling the nuclear spin of blood in a remainder of the plurality of arteries;
capturing one or more second MRI images of patient anatomy at the first imaging plane after a perfusion delay; and
comparing the one or more first and second MRI images to generate a map of perfusion associated with the at least one artery.

2. The method of claim 1, wherein the perfusion delay is 1-3 seconds after the step of applying a series of RF pulses.

3. The method of claim 1, wherein the steps of capturing one or more first MRI images, applying a series of RF pulses, applying a set of direct currents and capturing one or more second images are repeated for a second imaging plane.

4. The method of claim 1, wherein the coil array is a device placed on the patient's head.

5. The method of claim 1, wherein the coil array is a device placed on the patient's neck.

6. The method of claim 1, wherein the non-uniform magnetic field includes one or more regions where nuclear spins are at least 100 Hz off-resonance of the RF labeling field at the location of the remainder of the plurality of arteries.

7. The method of claim 1, further comprising applying a second set of direct currents to the plurality of coils in the coil array, such that a second resulting field in the first imaging plane is a magnetic field that is more uniform than without the second set of DC currents.

8. The method of claim 1, wherein the at least one artery includes a carotid artery.

9. The method of claim 1, wherein the at least one artery includes a vertebral artery.

10. A system for magnetic resonant imaging (MRI), comprising:
at least one RF coil configured to transmit an RF signal;
a coil array configured for placement on a patient in an MRI imaging system at a labeling plane that bisects a plurality of arteries; and
a computer configured to
capture one or more first MRI images of patient anatomy at a first imaging plane that is different from the labeling plane,
apply a series of RF pulses to the at least one RF coil, to create an RF labeling field in the labeling plane,
apply a first set of direct currents to a plurality of coils in the coil array while the RF pulses are applied, such that a first resulting field in the labeling plane is a non-uniform magnetic field such that the RF field labels the nuclear spin of blood in at least one artery of the plurality of arteries, while substantially not labeling the nuclear spin of blood in a remainder of the plurality of arteries,
capture one or more second MRI images of patient anatomy at the first imaging plane after a perfusion delay, and
compare the one or more first and second MRI images to generate a map of perfusion associated with the at least one artery.

11. The system of claim 10, wherein the perfusion delay is 1-3 seconds after the series of RF pulses are applied.

12. The system of claim 10, wherein computer is further configured to repeat the steps of capturing one or more first MRI images, applying a series of RF pulses, applying a set of direct currents and capturing one or more second images for a second imaging plane.

13. The system of claim 10, wherein the coil array is a device placed on the patient's head.

14. The system of claim 10, wherein the coil array is a device placed on the patient's neck.

15. The system of claim 10, wherein the non-uniform magnetic field includes one or more regions where nuclear spins are at least 100 Hz off-resonance of the RF labeling field at the location of the remainder of the plurality of arteries.

16. The system of claim 10, wherein computer is further configured to apply a second set of direct currents to the plurality of coils in the coil array, such that a second resulting field in the first imaging plane is a magnetic field that is more uniform than without the second set of DC currents.

17. The system of claim 10, wherein the at least one artery includes a carotid artery.

18. The system of claim 10, wherein the at least one artery includes a vertebral artery.

19. A method for perfusion magnetic resonant imaging (MRI) within an MRI system having at least one RF coil configured to transmit an RF signal, comprising:
providing a coil array configured for placement on a patient in the MRI system at a labeling plane that bisects a plurality of arteries;
capturing one or more first MRI images of patient anatomy at a first imaging plane that is different from the labeling plane;
applying a series of RF pulses to the at least one RF coil, to create an RF labeling field in the labeling plane;
applying a first set of direct currents to a plurality of coils in the coil array while the RF pulses are applied, such that a first resulting field in the labeling plane results in a labeling region where a nuclear spin of blood is more on-resonance with the RF labeling field than without the first set of DC currents;
capturing one or more second MRI images of patient anatomy at the first imaging plane after a perfusion delay; and
comparing the one or more first and second MRI images to generate a map of perfusion.

20. The method of claim 19, wherein the first resulting field in the labeling plane includes one or more regions where nuclear spins are at least 100 Hz off-resonance of the RF labeling field.
